(19) United States
(12) Patent Application Publication
(10) Pub. No.: US 2006/0002888 A1
(43) Pub. Date: Jan. 5, 2006

(54) Polyionenes for treating infections associated with cystic fibrosis

(75) Inventors: Richard J. Fitzpatrick, Marblehead, MA (US); Keith K. Shackett, Athol, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Genzyme Corporation, Cambridge, MA (US)

(21) Appl. No.: 11/132,150

(22) Filed: May 18, 2005

Related U.S. Application Data
(63) Continuation of application No. PCT/US03/36859, filed on Nov. 19, 2003.
(60) Provisional application No. 60/427,512, filed on Nov. 19, 2002.

(51) Int. Cl. A61K 31/785

(52) U.S. Cl. 424/78.3

(57) ABSTRACT

A method of using ionene polymers for the treatment or prevention of infections (e.g., pulmonary infections) in cystic fibrosis patients is provided. The method comprises administering to a mammal an effective amount of an ionene polymer to prophylactically or therapeutically treat infections associated with cystic fibrosis.

### RELATED APPLICATION

This application is a continuation of International Application No. PCT/US03/36859, which designated the United States, was filed on Nov. 19, 2003, and was published in English, which claims the benefit of U.S. Provisional Application No. 60/427,512, filed on Nov. 19, 2002. The entire teachings of the International Application and U.S. Provisional Application are hereby incorporated herein by reference.

### BACKGROUND OF THE INVENTION

Cystic fibrosis (CF) is a lethal autosomal recessive disorder, which affects about 30,000 people in the United States. As such, it is the most common fatal hereditary disorder for Caucasians in the United States. The average life expectancy for American CF patients is 31.3 years according to the U.S. Cystic Fibrosis Foundation Database for 1996. In South America, the median survival age remains at about 9 years.

CF is caused by one of several mutations in the cystic fibrosis transmembrane conductance regulator protein (CFTR). CFTR is synthesized mainly in epithelial cells in the respiratory passages, small intestine, pancreas and sweat glands and serous glands of the lung. Normal CFTR is transported to the cell surface after synthesis, where it regulates the flow of chloride in and out of the cell and influences sodium transport. In CF patients, the mutant CFTR does not reach the cell surface, which leads to excess sodium in the cells and tissues. The exact symptoms and severity of the disease vary depending on the specific mutation in CFTR. Sometimes the disease is diagnosed soon after birth, but other cases of CF may not be detected for months or years.

CF primarily affects the respiratory, digestive and reproductive systems, as well as the sweat glands. Patients suffering from CF produce large quantities of sweat and mucus, which is a response to the excess sodium in cells. The mucus secreted is very thick and responsible for many of the symptoms experienced by CF patients. In the digestive tract, the mucus often blocks pancreatic and gallbladder secretions, leading to difficulty digesting food and nutritional deficiencies. The mucus also often blocks the reproductive organs, particularly in males, where over 90% of males with CF are sterile.

The most serious effects of CF are seen in the respiratory tract. The thick mucus secretions block passageways in the lungs and sinuses, causing them to be susceptible to microbial infections. Respiratory tract infections, which lead to respiratory inflammation and eventually respiratory failure, are the primary cause of mortality in CF patients. The most common organisms to infect the respiratory tract are Pseudomonas bacteria, specifically P. aeruginosa. It is difficult to completely eradicate this bacterium, even with antibiotic treatment, so CF patients often have a pattern of colonization and subsequent low-grade persistent infection with periodic worsening and damaging inflammatory events.

In general, pathogenic colonization and infections are difficult to treat in cystic fibrosis patients. The pathogens that colonize the respiratory tracts of CF patients often develop resistance to pharmaceutical agents, such that the number of effective treatment options decreases with the age of the patient. Also, the viscous character of the mucus acts as a type of biofilm, thereby reducing the ability of the antimicrobial agents to penetrate through the mucus to reach the site of infection.

Present therapies for CF-associated infections are often inadequate, as pathogens develop resistance to various antibiotic or antimicrobial regimens. When continued infections cause irreversible tissue damage, CF patients must receive a lung transplant for continued survival. This is an expensive and risky procedure that relies on finding a donor. Therefore, there is a need for an improved antivirus, antiparasitic and/or antimicrobial agent that can treat the persistent infections encountered by CF patients. The antivirus, antiparasitic and/or antimicrobial agent should be effective against a broad range of organisms. The antivirus, antiparasitic and/or antimicrobial agent should also be slow to elicit resistance from pathogens.

### SUMMARY OF THE INVENTION

This invention discloses the use of polyionene polymers in preventing or inhibiting colonization and treating or preventing infections in patients suffering from cystic fibrosis. For example, the polyionenes poly(trimethylene dipyridine-alt-octane chloride) and poly(trimethylene dipyridine-alt-2,7-dihydroxyoctane chloride) have been found to be active against a broad range of pathogens (Example 19). In addition, these polyionenes are active against bacterial strains that are resistant to conventional antibiotics (Example 20). Moreover, resistance to these polyionenes evolves slowly (Example 21). Based on these discoveries, methods of preventing or inhibiting colonization and treating and/or preventing infection in a cystic fibrosis patient are disclosed.

The method of inhibiting colonization or treating or preventing infection (e.g., pulmonary infection) in a cystic fibrosis patient comprises administering to the patient an effective amount of an ionene polymer. A cystic fibrosis patient is at risk of colonization (the presence of pathogens) of the pulmonary system by various pathogens. In a preferred embodiment of the present invention, the ionene polymer comprises a repeat unit represented by Structural Formula (I):

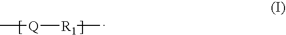

The polymer may be comprised of identical or non-identical repeat units so as to form either a homopolymer or a copolymer.

R₁ is a substituted or unsubstituted hydrocarbyl group. Preferably, R₁ is a substituted or unsubstituted arylene or lower alkylene group.

Each Q is represented by Structural Formula (II), (III), (IV), (V), or (VI):

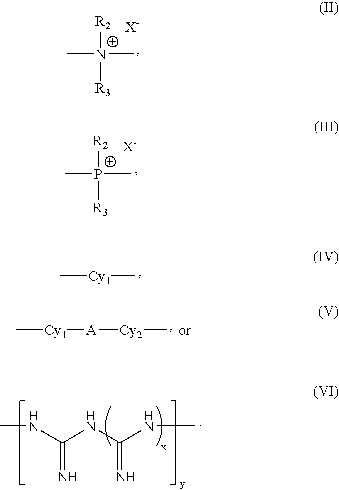

Cy₁ and Cy₂ are each independently a quaternary nitrogen-containing monocyclic heteroaromatic ring, a protonated tertiary nitrogen-containing non-aromatic heterocyclic ring or a quaternary nitrogen-containing non-aromatic heterocyclic ring.

A is a covalent bond, or a substituted or unsubstituted lower alkylene group.

R₂ and R₃ are independently —H or a substituted or unsubstituted aliphatic or aromatic group. Preferably, in the repeat units of Structural Formulas (II) and (III), R₂ and R₃ are each independently —H, an alkyl group or a hydroxyalkyl group.

Each X⁻, separately or taken together with other X⁻s, is a physiologically acceptable anion.

The values x and y are integers, where x is an integer from 0-4 or from 1-4 and y is an integer from 1-5 or from 2-5.

The present invention also provides the use of the ionene polymers disclosed herein in the manufacture of a medicament for the treatment or prevention of an infection in a cystic fibrosis patient.

The ionene polymers of the present invention have been found to be active against multiple organisms. Pathogenic resistance to these ionene polymers tends to evolve slowly. The ionene polymers of this invention additionally have been found to be low in toxicity to warm-blooded animals.

### BRIEF DESCRIPTION OF THE DRAWINGS

FIG. 1 shows the antimicrobial activity of two conventional antibiotics and 3 polyionenes in the diluted sputum of cystic fibrosis patients. The compound identified as 336-040-0003 is poly(trimethylene dipyridine-alt-octane) (TMDP-C₈). The compound identified as 456-069-0006 is poly(trimethylene dipyridine-alt-5-oxanonane). The compound identified as 461-170-0000 is a 3.5-mer of TMDP-C₈, where the terminal groups are each trimethylenedipyridine.

FIG. 2 shows that poly(trimethylene dipyridine-alt-5-oxanonane) (identified as 456-069-6) reduces the bacterial load in a chronic Pseudomonas aeruginosa infection model.

### DETAILED DESCRIPTION OF THE INVENTION

The present invention provides a method of using ionene polymers in pharmaceutical compositions for the treatment of infections associated with cystic fibrosis. “Ionene polymers” or “polyionenes,” as used in the present invention, are cationic polymers or copolymers with quaternary nitrogen or phosphorus (e.g., having four carbon atoms bound to the central nitrogen or phosphorus atom) or having a protonated secondary or tertiary nitrogen or phosphorus located in the main polymeric chain or backbone of the polymer, providing a positive charge. Polyionenes can also be polyguanidines or copolymers thereof, where the cationic nitrogen atom is an imide nitrogen directly bonded to the polymer backbone. Each polymer typically comprises from 50 to about 500 repeat units.

The present method includes administering a polymer or oligomer of the present invention to a CF patient before colonization or before an infection is acquired to prevent or inhibit onset of an infection. The method also includes treating a CF patient who is suffering from an active infection. Common infections include pulmonary infections.

Colonization and infections associated with CF are typically caused by a large variety of pathogens including Gram negative bacteria, Gram positive bacteria, fungi and viruses capable of infecting respiratory tract tissues. Bacteria and fungi associated with CF include, but are not limited to, species of Pseudomonas, Staphylococcus, Haemophilus, Burkholderia, Aspergillus, Candida, Mycobacteria, Mycoplasma, Stenotrophomonas, Escherichia, Achromobacter, Ralstonia, Acinetobacter, Streptococcus, Flavobacterium and Klebsiella. Specific microbial species causing the colonization or infection include Pseudomonas aeruginosa, Staphylococcus aureus, Haemophilus influenzae, Burkholderia cepacia, Aspergillusfumigatus, Candida albicans, Mycoplasma pneumoniae, Stenotrophomonas maltophiliai, Escherichia coli, Ralstonia mannitolilytica, Ralstonia pickettii, Streptococus pneumoniae, Flavobacterium indologenes, Burkholderia gladioli, Acineotobacter baumannii, Achromobacter xylosoxidans and Klebsiella pneumoniae. Viruses associated with CF include influenza virus (e.g., influenza virus A, influenza virus B, influenza virus C), respiratory syncytical virus and Rhinovirus. Pseudomonas aeruginosa is one of the most common infections occurring in CF patients and is advantageously treated or inhibited by the present method.

In a preferred embodiment of the present invention, Q is represented by Structural Formula (IV) and Cy₁ is a piperidinium ring having a quaternary nitrogen additionally substituted with a hydrogen or a substituted or unsubstituted lower alkyl group. More preferably, the quaternary nitrogen is additionally substituted with a lower alkyl or hydroxy substituted lower alkyl group. An example of a “piperidinium” ionene repeat unit is represented in Structural Formula (VII): 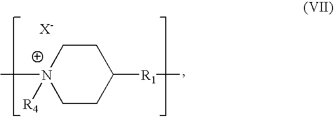
where R₄ is hydrogen or a substituted or unsubstituted lower alkyl group and R₁ is as defined above. A specific example of a piperidinium ionene repeat unit is shown in Structural Formula (VIII):

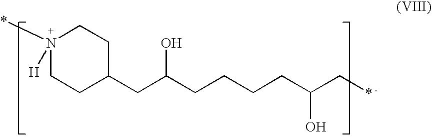

In another preferred embodiment, Q is represented by Structural Formula (V) and Cy₁ and Cy₂ are each piperidinium rings having a quaternary nitrogen additionally substituted independently with a hydrogen or a substituted or unsubstituted lower alkyl group and A is as defined above. More preferably, the quaternary nitrogen is additionally substituted with a lower alkyl or hydroxy substituted lower alkyl group. An example of a “piperidinium” ionene repeat unit of this type is represented in Structural Formula (IX): 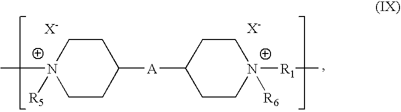
where A and R₁ are as defined above, and R₅ and R₆ are each independently hydrogen or a substituted or unsubstituted lower alkyl group. Preferably, R₅ and R₆ are each independently an alkyl group or a hydroxyalkyl group, and A is an unsubstituted straight chained lower alkylene group. Even more preferably, A is an unsubstituted straight chained lower alkylene group and R₁ is a substituted or unsubstituted straight chained lower alkylene or polyalkylene glycol group optionally substituted with one or more hydroxyl groups, preferably an unsubstituted polyalkylene glycol or —CH₂CHOH(CH₂)ₙCHOHCH₂— where n is an integer ranging from 0 to 8. Specific examples of “piperidinium” ionene repeat units are represented by the Structural Formulas (X), (XI), (XII), (XIII), (XIV), and (XV):

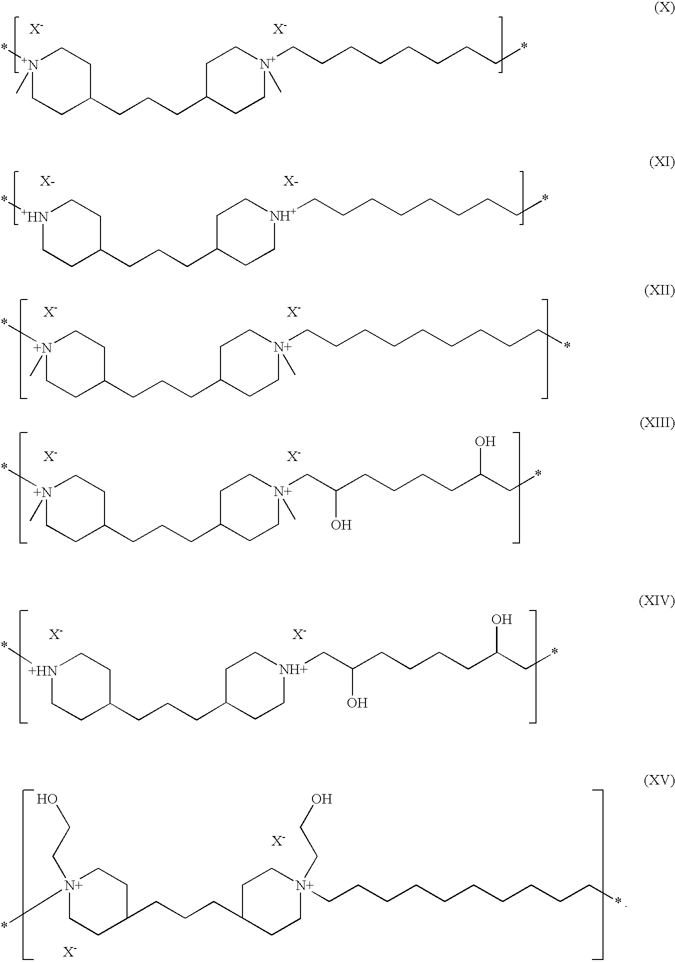

In yet another preferred embodiment, Q is represented by Structural Formula (V) and Cy₁ and Cy₂ are each pyridinium groups and A is as defined above. In one example of a “pyridinium” ionene polymer of this type, the polymer is characterized by repeat units represented by Structural Formula (XVI): 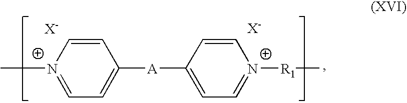
in which A and R₁ are as defined above. In a more preferred embodiment, A is an unsubstituted straight chained lower alkylene group. Even more preferably, A is an unsubstituted straight chained lower alkylene group and R₁ is a substituted or unsubstituted straight chained lower alkylene or polyalkylene glycol group optionally substituted with one or more hydroxyl groups, preferably an unsubstituted polyalkylene glycol or —CH₂CHOH(CH₂)ₙCHOHCH₂— where n is an integer ranging from 0 to 8. An example of a repeat unit with these components is represented by Structural Formula (XVII):

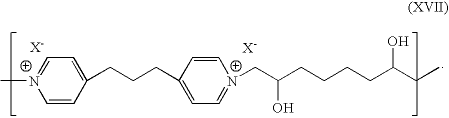

Other specific examples of “pyridinium” ionene polymers are represented by Structural Formulas (XVI), (XIX), (XX), (XXI), (XXII), (XXIII), and (XXIV): 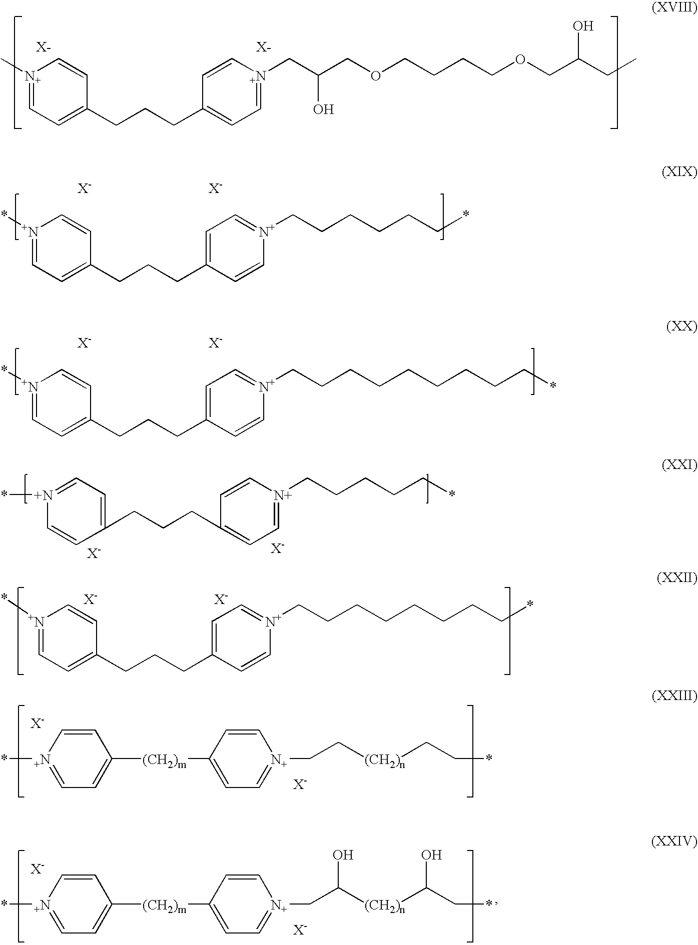
where m and n are independently-chosen integers ranging from 0 to 8. Typically, m is the same in each repeat unit and n is the same in each repeat unit.

Other specific examples of repeat units of polyionenes that can be used in the disclosed method are represented by Structural Formula (XXIII) above, wherein m is 1 and n is 0; m is 1 and n is 1; m is 1 and n is 2; m is 1 and n is 4; m is 1 and n is 5; m is 1 and n is 6; m is 1 and n is 8; m is 2 and n is 0; m is 2 and n is 1; m is 2 and n is 2; m is 2 and n is 4; m is 2 and n is 5; m is 2 and n is 6; m is 2 and n is 8; m is 3 and n is 0; m is 3 and n is 1; m is 3 and n is 2; m is 3 and n is 4; m is 3 and n is 5; m is 3 and n is 6; m is 3 and n is 8; m is 4 and n is 0; m is 4 and n is 1; m is 4 and n is 2; m is 4 and n is 4; m is 4 and n is 5; m is 4 and n is 6; m is 4 and n is 8; m is 5 and n is 0; m is 5 and n is 1; m is 5 and n is 2; m is 5 and n is 4; m is 5 and n is 5; m is 5 and n is 6; and m is 5 and n is 8.

Other specific examples of repeat units of polyionenes that can be used in the disclosed method are represented by Structural Formula (XXIV) above, wherein m is 1 and n is 0; m is 1 and n is 1; m is 1 and n is 2; m is 1 and n is 4; m is 1 and n is 5; m is 1 and n is 6; m is 1 and n is 8; m is 2 and n is 0; m is 2 and n is 1; m is 2 and n is 2; m is 2 and n is 4; m is 2 and n is 5; m is 2 and n is 6; m is 2 and n is 8; m is 3 and n is 0; m is 3 and n is 1; m is 3 and n is 2; m is 3 and n is 4; m is 3 and n is 5; m is 3 and n is 6; m is 3 and n is 8; m is 4 and n is 0; m is 4 and n is 1; m is 4 and n is 2; m is 4 and n is 4; m is 4 and n is 5; m is 4 and n is 6; m is 4 and n is 8; m is 5 and n is 0; m is 5 and n is 1; m is 5 and n is 2; m is 5 and n is 4; m is 5 and n is 5; m is 5 and n is 6; and m is 5 and n is 8.

One particular copolymer of the present invention comprises repeat units represented by Structural Formulas (XVII) and (XXII). Such copolymers can have alternating repeat units represented by Structural Formulas (XVII) and (XXII). Alternatively, such copolymers can comprise about 45-55% each of repeat units represented by Structural Formulas (XVII) and (XXII); about 30-40% of repeat units represented by Structural Formula (XVII) and about 60-70% of repeat units represented by Structural Formula (XXII); about 60-70% of repeat units represented by Structural Formula (XIII) and about 30-40% of repeat units represented by Structural Formula (XXII); about 23-27% of repeat units represented by Structural Formula (XVII) and about 73-77% of repeat units represented by Structural Formula (XXII); or about 73-77% of repeat units represented by Structural Formula (XVII) and about 23-27% of repeat units represented by Structural Formula (XXII). These copolymers can, for example, be block, alternating or random copolymers.

Another polyionene suitable for use in the present invention comprises a repeat unit where Q is represented by Structural Formula (II). When Q is represented by Structural Formula (II), R₁ is preferably a substituted or unsubstituted phenylene, lower alkylene, polyalkylene glycol group, or —CH₂CHOH(CH₂)ₙCHOHCH₂—, where n is an integer ranging from 0 to 8, and R₂ and R₃ are as defined above. Even more preferably, R₁ is a substituted or unsubstituted straight chained lower alkylene group or polyalkylene glycol optionally substituted with one or more hydroxyl groups.

Yet another polyionene suitable for use in the present invention comprises a repeat unit where Q is represented by Structural Formula (III). When Q is represented by Structural Formula (III), R₁ is preferably a substituted or unsubstituted arylene, lower alkylene, polyalkylene glycol group, or —CH₂CHOH(CH₂)ₙCHOHCH₂—, where n is integer ranging from 0 to 8, and R₂ and R₃ are as defined above. Even more preferably, R₁ is a substituted or unsubstituted straight chained lower alkylene group or polyalkylene glycol optionally substituted with one or more hydroxyl groups. A specific example is represented by Structural Formula (XXV):

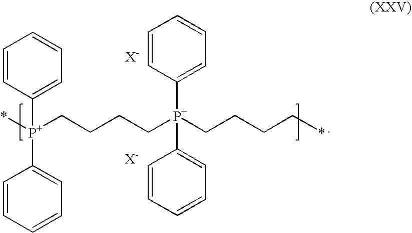

In another embodiment of the present invention, Q is represented by Structural Formula (VI). Preferably, R₁ is an unsubstituted lower alkylene or lower alkylene glycol group and x is 1 and y is 2; x is 1 and y is 3; x is 1 and y is 4; or x is 1 and y is 5. Specific examples of guanidine ionene polymers and copolymers comprise repeat units of formulas (XXVI), (XXVII), (XXVIII), and (XXIX):

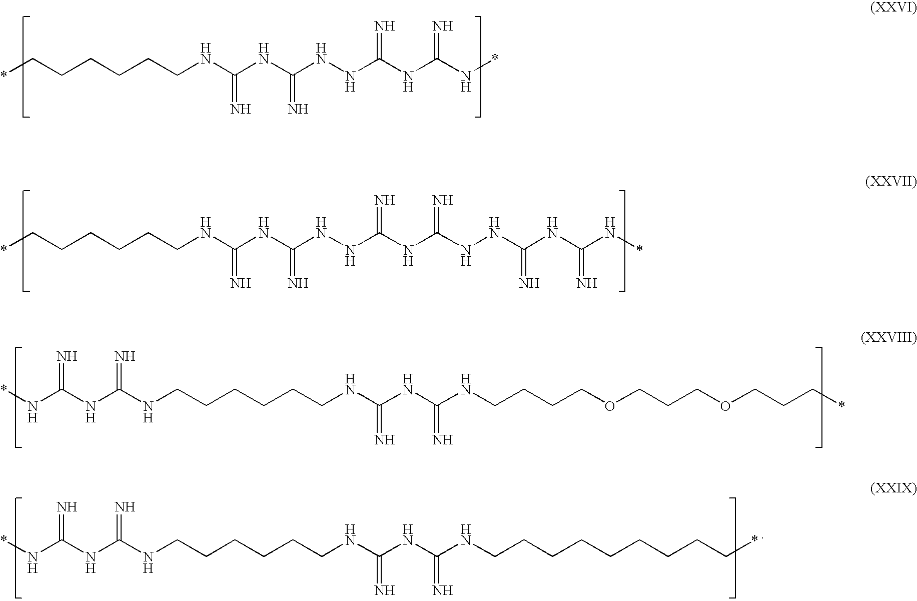

As noted above, ionene polymers suitable for use in the disclosed method include homopolymers and copolymers. The variables in each repeat unit of a copolymer of the present invention are independently selected. For example, in a copolymer, the alkylene group represented by A in one repeat unit can differ from the alkylene group represented by A in other repeat units. Alternatively, Q is identical in all repeat units and R₁ varies; R₁ is identical in all repeat units and Q varies; or Q and R₁ each vary among repeat units. In a homopolymer Q, R₁, and A are identical in all repeat units.

In one example of an ionene copolymer where Q varies within the polymer, Q is represented by Structural Formula (II) and Structural Formula (III). This copolymer is comprised of repeat units represented by Structural Formulas (XXXa) and (XXXb): 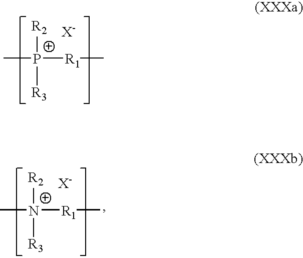
where R₁, R₂, R₃ and X are as defined above, and are chosen independently for each repeat unit. That is, R₁, R₂, R₃, and X are not necessarily the same throughout the copolymer.

In one example of an ionene copolymer of this type, the repeat units of Structural Formulae (XXXa) and (XXXb) alternate to form a repeat unit represented by Structural Formula (XXXI): 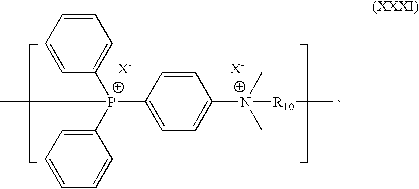
where R₁₀ is a substituted or unsubstituted lower alkylene group having 1 to about 24 carbon atoms, preferably having about 4 to about 12 carbon atoms. Each X⁻, separately or taken together with other X⁻s, is a physiologically acceptable anion.

In another example of an ionene copolymer where Q varies within the copolymer, Q alternates between repeat units represented by Structural Formulae (II)-(V), (X)-(XV), or (XVII)—(XXII) and a repeat unit represented by Structural Formula (VI). One copolymer of this type is represented by Structural Formula (XXXII):

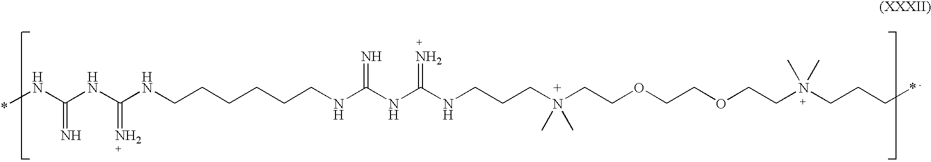

One example of a repeat unit of an ionene copolymer where Q is identical and R₁ varies is represented by Structural Formula (XXXIII):

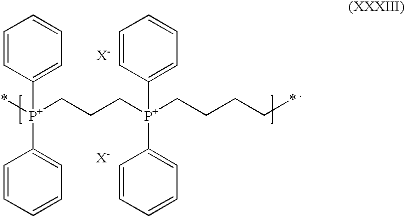

An “aliphatic group” is non-aromatic, consists solely of carbon and hydrogen and may optionally contain one or more units of unsaturation, e.g., double and/or triple bonds. An aliphatic group may be straight chained, branched, or cyclic and typically contains between about 1 and about 24 carbon atoms, more typically between about 1 and about 12 carbon atoms.

Aliphatic groups are preferably lower alkyl groups or lower alkylene groups, which include C1-24 (preferably C1-C12) straight chained or branched saturated hydrocarbons. An alkyl group is a saturated hydrocarbon in a molecule that is bonded to one other group in the molecule through a single covalent bond from one of its carbon atoms. Examples of lower alkyl groups include methyl, ethyl, n-propyl, iso-propyl, n-butyl, sec-butyl and tert-butyl. An oxyalkyl group is an alkyl group where an oxygen atom connects the alkyl group and one other group. An alkylene group is a saturated hydrocarbon in a molecule that is bonded to two other groups in the molecule through single covalent bonds from two of its carbon atoms. Examples of lower alkylene groups include methylene, ethylene, propylene, iso-propylene (—CH(CH₂)CH₂—), butylene, sec-butylene (—CH(CH₃)CH₂CH₂—), and tert-butylene (—C(CH₃)₂CH₂—).

Aromatic groups include carbocyclic aromatic groups such as phenyl, 1-naphthyl, 2-naphthyl, 1-anthracyl and 2-anthacyl, and heterocyclic aromatic groups such as N-imidazolyl, 2-imidazolyl, 2-thienyl, 3-thienyl, 2-furanyl, 3-furanyl, 2-pyridyl, 3-pyridyl, 4-pyridyl, 2-pyrimidyl, 4-pyrimidyl, 2-pyranyl, 3-pyranyl, 3-pyrazolyl, 4-pyrazolyl, 5-pyrazolyl, 2-pyrazinyl, 2-thiazolyl, 4-thiazolyl, 5-thiazolyl, 2-oxazolyl, 4-oxazolyl and 5-oxazolyl.

Aromatic groups also include fused polycyclic aromatic ring systems in which a carbocyclic aromatic ring or heteroaryl ring is fused to one or more other heteroaryl rings. Examples include 2-benzothienyl, 3-benzothienyl, 2-benzofuranyl, 3-benzofuranyl, 2-indolyl, 3-indolyl, 2-quinolinyl, 3-quinolinyl, 2-benzothiazolyl, 2-benzooxazolyl, 2-benzimidazole, 2-quinolinyl, 3-quinolinyl, 1-isoquinolinyl, 3-quinolinyl, 1-isoindolyl and 3-isoindolyl.

Phenyl is a preferred aromatic group.

“Arylene” is an aromatic ring(s) moiety in a molecule that is bonded to two other groups in the molecule through single covalent bonds from two of its ring atoms. Examples include phenylene (—(C₆H₄)—), thienylene (—(C₄H₂S)—) and furanylene (—(C₄H₂O)—).

A “nitrogen-containing non-aromatic heterocyclic group” is a cyclic group containing one or more nitrogen atoms in the ring, which can have one or more degrees of unsaturation provided that the group is not aromatic. Examples of nitrogen-containing non-aromatic heterocyclic groups include aziridine, azetidine, pyrrolidine, 2,3-1H-pyrrole, piperidine, morpholine, thiomorpholine, 1,2,3,4-tetrahydropyridine and 1,4-dihydropyridine.

A “polyalkylene glycol” is an alkylene group, which includes one or more ether linkages, where the chain includes a total of about 1 to about 12 carbon and oxygen atoms, and is optionally substituted with one or more hydroxyl groups. Preferably, the polyalkylene glycol is polyethylene glycol or polypropylene glycol.

A “hydrocarbyl group” is an alkylene or arylene group, i.e., —(CH₂)ₓ— or —(CH₂)ₓC₆H₄(CH₂)ₓ— where x is a positive integer (e.g., from 1 to about 30), preferably between 6 and about 30, more preferably between about 6 and about 15. The carbon chain of the hydrocarbyl group may be optionally interrupted with one or more ether (—O—), thioether (—S—), amine (—N⁺(R^{a})—), or ammonium (—N⁺(R^{a})(R^{b})—) linkages, or a combination thereof. R^{a} and R^{b} are independently —H, alkyl, substituted alkyl, phenyl, or substituted phenyl. R^{a} and R^{b} can be the same or different, but are typically the same. Examples of hydrocarbyl groups include butylene, pentylene, hexylene, heptylene, octylene, nonylene, decylene, dodecylene, 4-oxaoctylene, 5-oxanonylene, 4-azaoctylene, 4-thiaoctylene, 3,6-dioxaoctylene, 3,6-diazaoctylene, and 4,9-dioxadodecane.

Suitable substituents on an aliphatic, aromatic or benzyl group are those that do not substantially decrease the infection-treating or infection-preventing properties of the molecule. Examples of suitable substituents on an aliphatic, aromatic or benzyl group may include, for example, halogen (—Br, —Cl, —I and —F), —OR, —CN, —NO₂, —NR₂, —COOR, —CONR₂, —SOₖR (k is 0, 1 or 2) and —NH—C(═NH)—NH₂. An aliphatic group can also have ═O or ═S as a substituent. Each R is independently —H, an aliphatic group, a substituted aliphatic group, a benzyl group, a substituted benzyl group, an aromatic group or a substituted aromatic group, and is preferably —H, a lower alkyl group, a benzylic group or a phenyl group. Substituent groups can be selected such that all substituents are either neutral or positively charged. A substituted benzylic group or aromatic group can also have an aliphatic or substituted aliphatic group as a substituent. A substituted aliphatic group can also have a benzyl, substituted benzyl, aromatic or substituted aromatic group as a substituent. A substituted aliphatic, substituted aromatic or substituted benzyl group can have more than one substituent. A preferred substituent on an aliphatic group is —OH.

The anions represented by X⁻ in the polymer can be the same or different. Each X⁻ in a repeat unit can separately be a monovalent anion, i.e., an anion having a negative charge of one. Alternatively, two or more X⁻s in the same repeat unit or in different repeat units, taken together, can represent an anion having a negative charge of two, three or more. A polymer can comprise anions of different charges. Examples of suitable counteranions include sulfate, bisulfate, sulfite, bisulfite, phosphate, monohydrogenphosphate, dihydrogenphosphate, metaphosphate, pyrophosphate, chloride, bromide, iodide, acetate, proprionate, decanoate, caprylate, acrylate, formate, isobutyrate, caproate, heptanoate, propiolate, oxalate, malonate, succinate, fumarate, maleate, benzoate, alkyl sulfonate, phenylacetate, citrate, lactate, glycolate, tartrate, carbonate, bicarbonate and the like.

One anion can be exchanged for a second anion by various methods described in U.S. Application No. 60/397,868 and PCT Application No. PCT/US03/22514, the contents of which are incorporated herein by reference. In one such method, a proportion of the first anions of the ionene polymer can be exchanged for another anion by dissolving the polyionene in a solution containing the second anion or a mixture of the first and second anions. Another anion exchange method involves contacting the polyionene with an anionic exchange resin loaded with the desired second anion. Ion exchange processes involving an anionic exchange resin can be carried out in a throw-away mode, a regenerative mode, or in a continuous counter-current mode in simulated moving bed (SMB) equipment. In a further method, a proportion of the first anions of the polyionene can be exchanged for a second anion by electrodialysis. In electrodialysis, for example, a polyionene solution and a solution containing a salt having a desired second anion are passed through alternate channels of a stack of cation and/or anion exchange membranes. Conditions such as voltage, current density, flow rate of the solutions, and operation in co- or counter-current mode are controlled to produce a polyionene with the desired anion content. Polyionenes that have had their anions altered by any of the previously described methods can be purified by ultrafiltering the polyionene. Typically, ultrafiltration occurs simultaneously with or following anion exchange. For processes involving electrodialysis, ultrafiltration typically occurs prior to electrodialysis. Ultrafiltering a polyionene typically includes one or more cycles of diluting and concentrating the polyionene, whereby anions not bound to the polyionene and other contaminants are forced through a membrane and removed during concentration.

Also included in the present invention are physiologically acceptable salts of the polymers having guanidine repeat units or polymers comprising 1°, 2°, or 3° amines. Salts can be formed by reacting the polymer with a suitable acid. Examples include the corresponding acid of the counteranions listed above. Polymers having guanidine repeat units can have, for example, up to one molecule of hydrochloride or hydrobromide for every —NHC(═NH)NH— group or amine in the repeat unit.

The polymer can be administered alone or in a pharmaceutical composition comprising the polymer, a pharmaceutically acceptable carrier, and optionally, one or more additional drugs, e.g., antibiotics or antimicrobials. Examples of co-therapies for infections or complications due to CF include tobramycin and other aminoglycosides, ciprofloacin and other fluoroquinolones, rifabutin, ethambutol, clarithromycin, clofazimine, aztreonam, cephalothin, cefazolin, nafcillin, ticarcilin, clavulanate, gentamicin, amikacin, ceftazidime, piperacillin, imipenem, cefepime, chloramphenicol, colistin, dicloxacillin, cefaclor, amoxicillin, azithromycin, trmethoprim/sulfa, cefpodoxime, tetracyclines, amiloride and meropenem. These antibiotics can be administered orally, by injection or by pulmonary means. The term “pulmonary” as used herein refers to any part, tissue or organ whose primary function is gas exchange with the external environment, i.e., O₂/CO₂ exchange, within a patient. “Pulmonary” typically refers to the tissues of the respiratory tract. Thus, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli). For purposes of the present invention, “pulmonary” is also meant to include a tissue or cavity that is contingent to the respiratory tract, in particular, the sinuses.

The polymer can also be administered with an anti-inflammatory drug or steroid such as ibuprofen, prednisone (corticosteroid) or pentoxifylline. Another suitable co-therapy is administering dornase alfa (DNase), nacystelyn, gelsolin or hypertonic saline, which reduce mucus buildup, or administering a decongestant or bronchodilator (e.g, a beta adrenergic receptor agonist, an anticholinergic drug, theophylline).

The polymers of the present invention can also be administered following a physical therapy that aids mucus drainage. Such treatments include chest physiotherapy (manual or mechanical). Manual techniques include autogenic drainage and percussive techniques. Devices for mechanical therapy include positive expiratory pressure treatment, the “Flutter” mucus clearance device (a device that produces oscillations during exhalation), an inflatable vest driven by a pulsed-air delivery system.

The form in which the polymer is administered, for example, powder, tablet, capsule, solution, or emulsion, depends in part on the route by which it is administered. Polymers of the present invention are typically administered by pulmonary means, intranasally or orally, but can be administered parenterally.

Conventional means to deliver the active agent by pulmonary means a to patient include administration of an aerosol formulation containing the active agent from, for example, a manual pump spray, nebulizer or pressurized metered-dose inhaler.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

When administering the drug, the patient must actuate the drug delivery device. The actuation releases a fraction of the formulation from within the canister to the external environment. A force, created by vaporized propellant, expels the drug into the air and away from the device. The patient then inhales the aerosolized drug. The metering valve controls the amount of the formulation released, which, in turn, effectively controls the amount of drug available for inhalation by the patient.

Particles can also be administered by pulmonary means. To ensure that the drug particles have the proper size and shape, the particles may be analyzed using known techniques for determining particle morphology. For example, the particles can be visually inspected under a microscope and/or passed through a mesh screen. Preferred techniques for visualization of particles include scanning electron microscopy (SEM) and transmission electron microscopy (TEM). Particle size analysis may take place using laser diffraction methods. Commercially available systems for carrying out particle size analysis by laser diffraction are available from Clausthal-Zellerfeld, Germany (HELOS H1006).

Particles for pulmonary administration are typically substantially nonacicular particles. The particles will preferably have an average particle size in the range of about 0.5 micrometer to about 10 micrometer, more preferably in the range of about 1 micrometer to about 7.5 micrometer, and most preferably in the range of about 1 micrometer to about 5 micrometer. Preferably, greater than about 85%, more preferably greater than about 95%, and most preferably greater than about 98% of the population of particles in the formulation will fall within the desired particle size range, e.g., about 0.5 micrometer to about 10 micrometer, about 1 micrometer to about 7.5 micrometer, and so on.

Preferred drug delivery devices for particles are metered-dose inhalers. Metered-dose inhalers are described in Remington: The Science and Practice of Pharmacy, Twentieth Edition (Easton, Pa.: Mack Publishing Co., 2000) and in Ansel et al., Pharmaceutical Dosage Forms and Drug Delivery Systems, Sixth Edition (Malvern, Pa.: Lea & Febiger, 1995). The components of the drug delivery device, e.g., canister, housing, metering valve, etc., are commercially available. For example many components are available from 3M Corporation, St. Paul, Minn. Typically, although not necessarily, the amount of pharmaceutical formulation (including polymer, solvents and other expicipients) that is released per actuation of the drug delivery device is about 5 micrograms to about 100,000 micrograms of formulation.

Suitable carriers and diluents for an ionene polymer will be immediately apparent to persons skilled in the art. These carrier and diluent materials, either organic or inorganic in nature, include, for example, gelatin, lactose, starch, magnesium stearate, preservatives (stabilizers), sugars, emulsifying agents, salts and buffers. Other pharmaceutically acceptable carriers include, for example, commercially available inert gels, or liquids supplemented with albumin, methyl cellulose, or a collagen matrix.

An effective amount of an ionene polymer to be administered will be determined on an individual basis, and will be determined at least in part, by consideration of the individual's size, the severity and type of the infection to be treated or prevented and the result sought. As used herein, an effective amount refers to an appropriate amount of ionene polymer, which results in a desired therapeutic or prophylactic effect with respect to infection stemming from cystic fibrosis, as defined above. Typical dosages for inhaled, applied and/or ingested ionene polymers range from between about 0.05 μg/kg body weight to about 500 mg/kg body weight, more typically between about 0.1 μg/kg body weight to about 100 mg/kg body weight and even more typically between about 0.5 μg/kg body weight and about 10 mg/kg body weight.

The method is preferably used with human patients, but can also be used with other mammals, such as companion animals (e.g., dogs, cats, and the like), farm animals (horses, cattle, goats, and the like) and laboratory animals (hamsters, mice, rats, and the like).

Ionene polymers of the present invention can be prepared by a reacting a divalent electrophile such as an α,ω-dihalogenated alkane or a corresponding diepoxide with a divalent nucleophile such as 4,4′-trimethylenedipiperidine or N,N,N′,N′-tetramethyl-1,3-propanediamine. When preparing a polyguanidine, the divalent nucleophile is an α,ω-diaminoalkane or an α,ω-aminoguanidine and the divalent electrophile typically is an α,ω-biscyanoguanidine. Polymerizing with one divalent electrophile and one divalent nucleophile results in a homopolymer. Polymerizing with two or more divalent electrophiles and/or divalent nucleophiles results in a copolymer. Such homopolymers and copolymers are encompassed within the present invention.

Polyionene polymers are typically “capped” at the termini with a partially reacted divalent electrophile or nucleophile or a monovalent electrophile or nucleophile. For example, when polymerizing 4,4′-trimethylenepyridine and 1,6-dibromohexane (or the corresponding epoxide), the resulting polymer is capped at either end with one of the following groups: 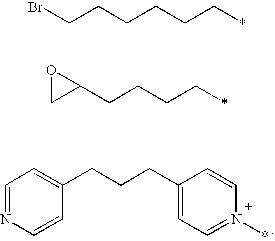
Optionally, the capping group can be reacted further, for example, by hydrolyzing the epoxide or reacting the halide or epoxide with a nucleophile. An example of a capping group for polyguanidine polymers or copolymers is represented by Structural Formula (XXXIV): 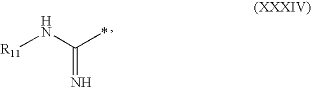
where R₁₁ is a C2-C90 alkyl, C2-C90 oxyalkyl, or aromatic group and the symbol “*” represents the bond connecting the cap to the polymer or copolymer.

Ionene polymers of the invention may also be cross-linked with primary, secondary or other polyfunctional amine using means known in the art. Ionene polymers can be cross-linked by polymerizing in the presence of a multivalent nucleophile (i.e., a compound with three or more nucleophilic groups such as a triamine or tetraamine) or a multivalent electrophile (i.e., a compound with three or more nucleophilic groups such as a trihalide or tetrahalide).

While this invention has been particularly shown and described with references to preferred embodiments thereof, it will be understood by those skilled in the art that various changes in form and details may be made therein without departing from the scope of the invention encompassed by the appended claims. The invention will now be further and specifically described by the following non-limiting Examples.

### EXEMPLIFICATION

### Example 1

### Preparation of poly(hexamethylenebiscyanoguanidine-alt-4,9-dioxadodecane) (XXVII)

Hexamethylenebiscyanoguanidine (3.99 mmoles, 1.00 g) and 4,9-dioxa-1,12-dodecanediamine (3.99 mmoles, 0.848 ml) were added to a 40 ml vial with a septa-cap followed by 2 equivalents of concentrated HCl. The mixture was heated to 135-145° C. in a shaker overnight. The resulting clear yellow, brittle solid was dissolved in water and purified by centrifugation through a 3K Macrosep filtration membrane.

### Example 2

### Preparation of poly(4,4′-trimethylenebis(1-methylpiperidinium)-alt-octane) (X)

4,4′-Trimethylenebis(1-methylpiperidine)-alt-1,8-Dibromooctane was prepared by dissolving 4,4′-Trimethylenebis(1-methylpiperidine) (39.9 ml) in 30 ml of DMF in a 250 ml Erlenmeyer flask. 1,8-Dibromooctane (27.63 ml) was also added to the flask. The reaction was purged with nitrogen, covered with a septum, and stirred with a magnetic stir plate. The initial solution was clear. After approximately 20 minutes of stirring the reaction exothermed and solidified. A light yellow solid polymer formed and was left to further polymerize for a week. The polymer was dissolved in ˜300 ml of deionized water and dialyzed (3500 molecular weight cut-off) in water 3× and 1× in water/MeOH 70%/30%.

### Example 3

### Preparation of poly(4-(dimethylamino)phenyldiphenylphosphonium-alt-dodecane) (XXXI, where R₁₀ is dodecyl)

4-(Dimethylamino)phenyldiphenylphosphine (1.73 mmoles, 0.529 g) and 1,12-dibromododecane (1.73 mmoles, 0.569 g) were dissolved in DMF (1 ml) and shaken for 1 week. The resulting viscous liquid was diluted with water and purified by centrifugation through a 3K Macrosep.

### Example 4

### Preparation of poly(4,4′-trimethylenedipyridinium-alt-hexane) (XIX)

4,4′-Trimethylenedipyridine (3.46 mmoles, 0.687 g) was added to a 40 ml vial followed by 2.3 ml of DMF/methanol (1:1 v:v). 1,6-dibromohexane (3.46 mmoles, 0.533 ml) was added and the vial was capped with a septa-cap. The vial was purged with nitrogen and placed in a shaker for 1 week. The resulting clear orange viscous solution was diluted in water and purified by centrifugation through a 3K Macrosep.

### Example 5

### Preparation of poly(4,4′-trimethylenedipyridinium-alt-nonane) (XX)

4,4′-Trimethylenedipyridine (3.46 mmoles, 0.687 g) was added to a 40 ml vial followed by 2.3 ml of DMF/methanol (1:1 v:v). 1,9-dibromononane (3.46 mmoles, 0.705 ml) was added and the vial was capped with a septa-cap. The vial was purged with nitrogen and placed in a shaker for 1 week. The resulting light orange waxy solid was dissolved in water and purified by centrifugation through a 3K Macrosep.

### Example 6

### Preparation of poly(N,N-dimethylpropylammonium-alt-N,N-dimethylhexylammonium)

N,N,N′,N′-Tetramethyl-1,3-propanediamine-alt-1,6-Dibromohexane was prepared by dissolving N,N,N′,N′-Tetramethyl-1,3-propanediamine (31.9 ml) in 40 ml of DMF in a 250 Erlenmeyer flask. 1,6-Dibromohexane (29.3 ml) was added to the flask. The reaction was purged with nitrogen, covered with a septum, and stirred with a magnetic stir plate. The initial solution was clear. A very quick reaction that exothermed and solidified occurred. An off white solid polymer formed and was left to further polymerize for a week. The polymer was dissolved in approximately 300 ml of deionized water and dialyzed (3500 MW) in water 3× and 1× in water/MeOH 70%/30%.

### Example 7

### Preparation of poly(hexamethylene biscyano guanidine-alt-nonane) (XXIX)

Hexamethylenebiscyanoguanidine (3.99 mmoles, 1.00 g) and 1,9-diaminononane (3.99 mmoles, 0.623 g) were added to a 40 ml vial with a septa-cap followed by 2 equivalents of concentrated HCl. The mixture was heated to 135-145° C. in a shaker overnight. The solid was dissolved in water and purified by centrifugation through a 3K Macrosep filtration membrane.

### Example 8

### Preparation of poly(4,4′-trimethylenedipiperidinium-alt-hexane) (XI)

4,4′-Trimethylenedipiperidine (3.466 mmoles, 1.139 g) was added to a 40 ml vial followed by 2 ml DMF/MeOH (1:1v:v). 1,6-Dibromohexane (3.466 mmoles, 0.533 ml) was added and the vial was capped with a septa-cap. The vial was purged with nitrogen and placed in a shaker for 1 week. The resulting opalescent waxy solid was dissolved in water and purified by centrifugation through a 3K Macrosep.

### Example 9

### Preparation of poly(hexamethylenebiscyanoguanidine-alt-hydrazine) (XXVI)

Hexamethylene biscyano guanidine (4.00 mmoles, 1.00 g) and hydrazine (4.00 mmoles, 0.274 g) were added to a 40 vial with a septa-cap followed by 2 equivalents of concentrated HCl. The mixture was heated to 165° C. in an oil-bath for 3 h. The resulting pink foam was acidified with 2 equivalents concentrated HCl, dissolved in water and purified by centrifugation through a 3K Macrosep filtration membrane.

### Example 10

### Preparation of poly(4-(dimethylamino)phenyldiphenylphosphonium-alt-nonane) (XXXI, where R10 is nonyl)

4-(Dimethylamino)phenyldiphenylphosphine (1.73 mmoles, 0.529 g) and 1,9-dibromononane (1.73 mmoles, 0.352 g) were dissolved in DMF (1 ml) and shaken for 1 week. The resulting viscous liquid was diluted with water and purified by centrifugation through a 3K Macrosep.

### Example 11

### Preparation of poly(4-(dimethylamino)phenyldiphenylphosphonium-alt-decane) (XXXI, where R10 is decyl)

4-(Dimethylamino)phenyldiphenylphosphine (1.73 mmoles, 0.529 g) and 1,10-dibromodecane (1.73 mmoles, 1.04 g) were dissolved in DMF (1 ml) and shaken for 1 week. The resulting viscous liquid was diluted with water and purified by centrifigation through a 3K Macrosep.

### Example 12

### Preparation of poly(hexamethylene biscyano guanidine-alt-1,3-aminoguanidine) (XXVII)

Hexamethylene biscyano guanidine (4.00 mmoles, 1.00 g) and 1,3-aminoguanidine (4.00 mmoles, 0.502 g) were added to a 40 ml vial with a septa-cap followed by 2 equivalents of concentrated HCl. The mixture was heated to 165° C. in an oil-bath for 3 h. The resulting orange solid was acidified with 1 eq. concentrated HCl, dissolved in water and purified by centrifugation through a 3K Macrosep filtration membrane.

### Example 13

### Preparation of poly(1,3-bis(diphenylphosphonium)propane-alt-butane) (XXXIII)

1,3-Bis(diphenylphosphino)propane (1.33 mmoles, 0.550 g) and 1,4-dibromobutane (1.33 mmoles, 0.159 g) were dissolved in DMF (0.769 ml) and shaken for 1 week. The resulting viscous liquid was diluted with water and purified by centrifugation through a 3K Macrosep.

### Example 14

### Preparation of poly(4-(dimethylamino)phenyldiphenylphosphonium-alt-butane) (XXXI, where R₁₀ is butyl)

4-(Dimethylamino)phenyldiphenylphosphine (1.73 mmoles, 0.529 g) and 1,4-dibromobutane (1.73 mmoles, 0.207 g) were dissolved in DMF (1 ml) and shaken for 1 week. The resulting viscous liquid was diluted with water and purified by centrifugation through a 3K Macrosep.

### Example 15

### Preparation of poly(1,4-bis(diphenylphosphonium)butane-alt-butane) (XXV)

1,4-Bis(diphenylphosphino)butane (2.31 mmoles, 0.986 g) and 1,4-dibromobutane (2.31 mmoles, 0.276 g) were dissolved in DMF (1.333 ml) and shaken for 1 week. The resulting viscous liquid was diluted with water and purified by centrifugation through a 3K Macrosep.

### Example 16

### Preparation of Crosslinked Polymers—Post-Polymerization Crosslinking

Hydroxyl-containing polymer (XVII) was cross-linked with 6 mole % 1,6-diisocyanatohexane in DMF to produce a gel. The gel was washed with 70% methanol-water and lyophilized.

### Example 17

### Preparation of Crosslinked Polymers—In Situ Crosslinking

N,N,N′,N′-Tetramethyl-1,3-propanediamine (34.64 mmoles, 5.795 ml), 1,9-dibromononane (34.64 mmoles, 7.048 ml), and 1,3,5-tris(bromomethyl)-2,4,6-trimethylbenzene (3.464 mmoles, 1.383 g) were dissolved in DMF (1 ml) and shaken for a week at room temperature. The resulting white gel was washed with hot DMF, methanol, and water and lyophilized.

### Example 18

### Preparation of poly(trimethylenedipyridinium-alt-2,7-dihydroxyoctane) (XVI)

Trimethylenedipyridine (100 g) was placed in a roundbottom flask. To the flask was added 1,2,7,8-diepoxyoctane (71.72 g). The reaction was stirred under nitrogen at room temperature for 20 min. until nearly all the trimethylenedipyridine was dissolved. At this time, acetic acid (121 g) was slowly added dropwise over a 24 hr period. The reaction was stirred at room temperature for an additional four days. The resulting material was dark blue and highly viscous. The solid was dissolved in water and purified by tangential flow with a 1K MWCO membrane.

### Example 19

### Antimicrobial Activity of Polyionenes

About 1-10 kDa Poly(trimethylene dipyridine-alt-octane chloride) (TMDP-C₈) and about 1.2-9 poly(trimethylene dipyridine-alt-2,7-dihydroxyoctane chloride) (TMDP-C₈(OH)₂) have broad, though not identical antimicrobial activity. They have been tested extensively against about 50 different strains of bacteria and Candida spp., and against most strains, minimum inhibitory concentrations (MIC's) do not vary by more than 2-fold (the limit of reproducible accuracy of the MIC broth dilution assay). Some representative activities are shown in Tables 1, 2 and 3.

TABLE 1
MIC (μg/ml) Against Selected Pathogens

| | Streptococcus | E. coli | Candida | | P. aeruginosa | |
|---|---|---|---|---|---|---|
| | spp. (9) | (2) | spp. (8) | A. baumannii | (5) | B. cepacia |
| TMDP-C₈ | 0.2-6.25 | 0.8 | 0.4-3.0 | 3.12 | 1.6-3.2 | 6.4 |

(Numbers in parentheses indicate numbers of species/strains tested.)

TABLE 2
Susceptibility of Bacteria to Polyionenes

| | | MIC (μg/mL) | |
|---|---|---|---|
| Organism | Strain ID | TMDP-C₈(OH)₂ | TMDP-C₈ |
| Acinetobacter | U.S. Army Inst. | 50 | 3.2 |
| baumannii | Surg. Res. | | |
| | 980112001 | | |
| Aeromonas | ATCC 35654 | 1.6 | 1.6 |
| hydrophila | | | |
| Branhamella | ATCC 25238 | 0.05 | 0.2 |
| catarrhalis | | | |
| Burkholderia | ATCC 25416 | >50 | 6.3 |
| cepacia | | | |
| Corynebacterium | ATCC 27010 | 0.05 | 0.1 |
| diphtheriae | | | |
| Enterococcus | ATCC 29212 | 0.05 | 0.4 |
| faecalis | | | |
| Enterococcus | ATCC 19434 | 0.05 | 0.8 |
| faecium | | | |
| Escherichia | ATCC 25922 | 1.6 | 0.8 |
| coli | | | |
| Haemophilus | ATCC 33391 | 6.3 | 1.6 |
| influenzae | | | |
| Klebsiella | ATCC 13883 | 1.6 | 1.6 |
| pneumoniae | | | |
| Listeria | ATCC 33090 | 0.4 | 0.4 |
| innocua | | | |
| Neisseria | ATCC 13077 | 0.2 | 0.4 |
| meningitidis | | | |
| Neisseria | ATCC 19695 | 0.8 | 0.1 |
| mucosa | | | |
| Neisseria sicca | ATCC 9913 | 0.1 | 0.8 |
| Neisseria | ATCC 49275 | 0.1 | 0.1 |
| subflava | | | |
| Pseudomonas | ATCC 27853 | 1.6 | 1.6 |
| aeruginosa | | | |
| Proteus | U.S. Army Inst. | 6.3 | 6.3 |
| mirabilis | Surg. Res. | | |
| | 770822034 | | |
| Rhodococcus | ATCC 6939 | 0.8 | 0.8 |
| equi | | | |
| Serratia | ATCC 13880 | 1.6 | 1.6 |
| marcescens | | | |
| Staphylococcus | ATCC 29213 | 0.2 | 0.8 |
| aureus | | | |
| Streptococcus | C946, Univ. | 0.05 | ND |
| aureus | Brit. Columbia | | |
| Staphylococcus | ATCC 14990 | 0.1 | 0.4 |
| epidermidis | | | |
| Staphylococcus | Genzyme Corp. | 0.05 | 0.4 |
| epidermidis | 14990 | | |
| Staphylococcus | ATCC 27844 | 0.05 | 0.1 |
| hominis | | | |
| Stenotrophomonas | ATCC 13637 | 0.8 | 0.8 |
| maltophilia | | | |
| Streptococcus | ATCC 49446 | 1.6 | 0.8 |
| agalactiae | | | |
| Streptococcus | ATCC 12959 | 6.31 | 0.8 |
| Group D | | | |
| Streptococcus | ATCC 6249 | >50 | 1.6 |
| mitis | | | |
| Streptococcus | ATCC 49456 | 12.5 | 1.6 |
| mitis | | | |
| Streptococcus | ATCC 25175 | 0.1 | 0.2 |
| mutans | | | |
| Streptococcus | ATCC 9811 | >50 | 3.2 |
| oralis | | | |
| Streptococcus | ATCC 55229 | >50 | 1.6 |
| oralis | | | |
| Streptococcus | ATCC 33400 | 12.5 | 1.6 |
| pneumoniae | | | |
| Streptococcus | ATCC 12344 | 0.4 | 0.2 |
| pyogenes | | | |
| Streptococcus | ATCC 13419 | 0.1 | 0.4 |
| salivarius | | | |
| Streptococcus | ATCC 10556 | <0.05 | 0.1 |
| sanguis | | | |

TABLE 3
Susceptibility of Candida Species to Polyionenes

| | Strain | MIC (μg/mL) | |
|---|---|---|---|
| Organism | Identification | TMDP-C₈(OH)₂ | TMDP-C₈ |
| Candida albicans | ATCC 18804 | 0.4 | 0.1 |
| Candida albicans | 4090, Univ. | 0.4 | 0.4 |
| | TX Hlth. Sci. | | |
| | Ctr. San Antonio | | |
| | (UTHSCSA) | | |
| Candida albicans | 4111, UTHSCSA | 0.4 | 0.4 |
| Candida albicans | 4227, UTHSCSA | 0.8 | 0.2 |
| Candida albicans | 4507, UTHSCSA | 0.8 | 0.4 |
| Candida dublinensis | 4116, UTHSCSA | 0.4 | 0.4 |
| Candida glabrata | ATCC 90030 | 0.4 | 0.4 |
| Candida glabrata | 4233, UTHSCSA | 0.4 | 0.8 |
| Candida glabrata | 4760, UTHSCSA | 0.4 | 0.4 |
| Candida glabrata | 4758, UTHSCSA | 0.4 | 0.4 |
| Candida krusei | ATCC 2340 | 0.2 | 0.2 |
| Candida krusei | 4566, UTHSCSA | 0.2 | 0.4 |
| Candida krusei | 4835, UTHSCSA | 0.2 | 0.4 |
| Candida lusitaniae | ATCC 34449 | 0.1 | 0.4 |
| Candida lusitaniae | ATCC 42720 | 0.2 | 0.4 |
| Candida parapsilosis | 6196, UTHSCSA | 0.2 | 0.2 |
| Candida tropicalis | ATCC 1369 | 0.1 | 0.4 |
| Candida tropicalis | 4305, UTHSCSA | 0.2 | 0.4 |

### Example 20

### Susceptibility of Antibiotic-Resistant Bacteria to Polyionenes

About 1-10 kDa TMDP-C₈ and about 1.2-9 kDa TMDP-C₈(OH)₂ have also been tested against bacterial strains resistant to conventional antibiotics. Antimicrobial activity against methcillin-resistant S. aureus, vancomycin-resistant Enterococcus spp., glycopeptide-resistant S. aureus and multiply resistant P. aeruginosa, Stenotrophomonas maltophilia and Acinetobacter spp. were within a 2-fold dilution of those obtained using antibiotic-susceptible strains of the same species (data not shown). This suggests that mechanisms of action of antimicrobial polymers differ from those of conventional antibiotics.

### Example 21

### Development of Antibiotic Resistance

We have performed in vitro resistance selection studies using 4 different classes of antimicrobial polymers (about 1-10 kDa biguanides, about 1-10 kDa phosphonium ionenes, dipiperidine ionenes (about 1-10 kDa TMBDP-C₈ and about 1.2 and 5 kDa dipyridine ionenes) against 5 bacterial strains (E. coli (ATCC 25922 or ATCC 11775), E. faecium (ATCC 19434) E. faecalis (ATCC 29212), S. aureus (ATCC 29213) or P. aeruginosa (ATCC 27853)). To select for resistance, ATCC strains were passaged 20-25 times in vitro in the presence of sub-inhibitory concentrations of the selecting compound. Isolates from each passage were then tested in an MIC assay for susceptibility to the selecting compound, to related non-peptide antimicrobial polymers and to conventional antibiotics. For these studies, resistance was defined as a change of ≧4-fold in the MIC.

Results from resistance evolution studies showed that for the biguanide and phosphonium ionene classes, there was no change in susceptibility over 25 passages for S. aureus, E. coli. P. aeruginosa or E. faecium. For TMBDP-C₈ (S. aureus) and TMDP-C₈(OH)₂, (P. aeruginosa) resistance emerged after 14-16 passages (in independent studies). This time course of resistance evolution is comparable to that seen for antimicrobial peptides, although published studies were run for only 7-15 passages. For comparison, those studies also examined resistance evolution to norfloxacin and gentamicin. Against P. aeruginosa, the MIC of norfloxacin rose 10-fold and that of gentamicin 190-fold within 11 passages. Against MRSA, the MIC of norfloxacin rose 85-fold over 15 passages.

### Example 22

### Activity of Ionenes in Infected Wounds

The non-substituted C₈-containing compound TMDP-C₈ (1-10 kDa) was tested at 10 mg/ml for its ability to reduce S. aureus infections introduced into dermal wounds in pigs. Compared with controls, treatment reduced recoverable microbial load by 4 logarithmic units; in this model, reduction by ≧1 log unit is considered significant. In parallel studies examining wound healing (in the absence of introduced microbial infection), treatment with TMDP-C₈ did not inhibit or retard healing. These 2 preliminary studies suggest that the cationic compound retains antimicrobial activity in the context of tissue/tissue exudate, and that at least when topically applied, does not appear to inhibit wound healing.

### Example 23

### Lowering of Oral Microbial Load

Studies were also conducted to assess the effect of treatment using 1.2-9 kDa TMDP-C₈(OH)₂ on oral microbial load in a hamster model of irradiation-induced oral mucositis. Following irradiation, animals were dosed 3× daily into the left cheek pouch with 15 mg/kg TMDP-C₈(OH)₂. Microbial samples were collected one hour after the final treatment on Days 8, 14 and 20 following irradiation, corresponding to different phases of disease. At each time point, total microbial load was reduced by approximately 1-2 logarithmic units. (In this model, this dose administration was shown to reduce ulceration by ˜80%).

### Example 24

### Antimicrobial Activity of Polyionenes in Sputum from CF Patients

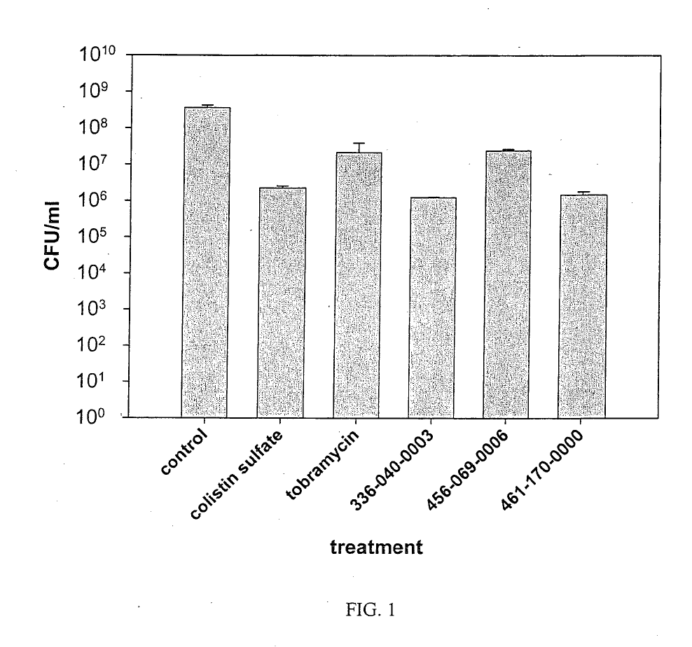

The methods used in this example were modified from those of Sajjan and coworkers (U. Sajjan, et al., “P113D, An antimicrobial peptide active against Pseudomonas aeruginosa, retains activity in the presence of sputum from cystic fibrosis patients,” Antimicrobial Agents and Chemotherapy 45(12): 3437-3444). Briefly, sputum specimens from 5 cystic fibrosis (CF) patients was pooled, aliquotted at 250 μl, and stored at −80° C. until use. For each experiment, sputum was thawed, was diluted 1:10 in 0.85% sterile saline and was incubated for 1 hr at 35° C. in the presence of 100 μg/ml Dornase Alpha. Test compounds were then added to a final concentration of 100× the Minimum Inhibitory Concentration (range: 100-600 μg/ml), and samples were incubated for 6 hr at 35° C. Ten-fold serial dilutions were then prepared, plated on Tryptic soy agar medium, incubated for 48 hr at 35° C., and colonies were enumerated. The results are shown in FIG. 1. Colistin sulfate and tobramycin, antibiotics commonly used to treat CF patients, are included for comparison. The three polyionenes tested, poly(TMDP-C₈), a 3.5-mer of TMDP-C₈ and poly(trimethylene dipyridine-alt-5-oxanonane), exhibited antimicrobial activity similar to that of the two common antibiotics.

In separate studies, this pool of patient sputum specimens was shown to contain Pseudomonas aeruginosa, Alcaligenesfaecalis, Acinetobacter haemolyticus and CDC group VB3, in addition to other uncharacterized species.

### Example 25

### Antimicrobial Activity of Polyionenes in Rat CF Models

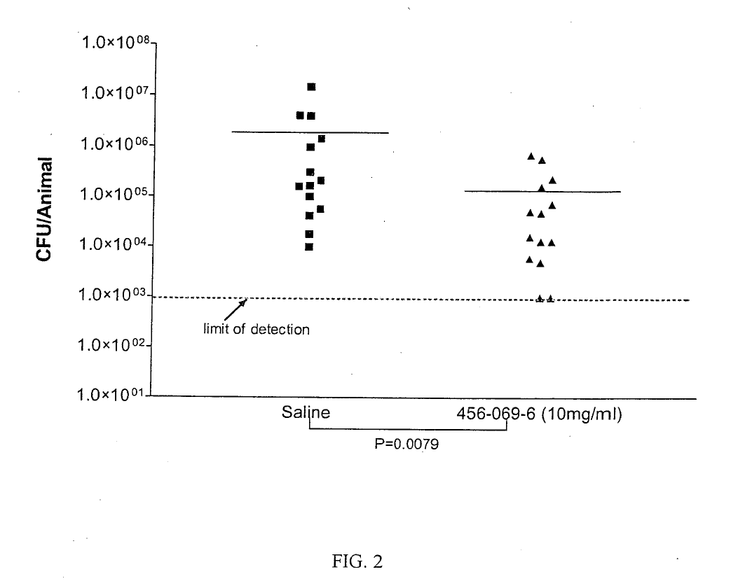

Male Sprague-Dawley rats were inoculated with 7×10⁴ CFU Pseudomonas aeruginosa by intratracheal instillation into the lungs, according to the model developed by Hash and coworkers (Cash, H. A., et al., “A rat model of chronic respiratory infection with Pseudomonas aeruginosa,” Am. Rev. Respir. Dis. 119:453-459 (1979). Briefly, P. aeruginosa was embedded in agarose beads of approximately 30 micron diameter in a volume of 100 ul for the inoculum. A 10 mg/mL solution of poly(trimethylene dipyridine-alt-5-oxanonane (indicated as 456-069-6 in FIG. 2) or saline was administered daily by intranasal delivery of 100 μL of one of the solutions on days 3 through 6 post-infection. Bacterial load was determined by serial dilution and culture of lung homogenates on day 6. The results are shown in FIG. 2. The arithmetic mean for colony forming units in rats receiving saline was 1.89×10⁶, while rats receiving the polyionene had an arithmetic mean of 1.35×10⁵ CFUs. A similar reduction was seen in the geometric means of CFUs among the rat population treated with the polyionene, from 2.87×10⁵ to 2.82×10⁴. The difference in number of CFUs among treatment groups was significant, with a p value of 0.0079.

### Example 26

### Safety and Efficacy of Poly(trimethylene dipyridine-alt-5-oxanonane)

Poly(trimethylene dipyridine-alt-5-oxanonane) having a molecular weight of 0.5 to 1 kDa was tested in A-549 lung epithelial cells to determine its IC₅₀ value in comparison with tobramycin, a conventional antibiotic. In addition, the minimum inhibitory concentrations for the two compounds were determined in vitro for a number of bacterial strains and species. The results are as shown below, where all concentrations are expressed as μg/mL:

| | | | P. aeruginosa | P. aeruginosa | | |
|---|---|---|---|---|---|---|
| Compound | IC₅₀ | S. aureus | G38 | PA01 | B. cepacia | S. maltophilia |
| Tobramycin | 5000 | 0.195 | 0.39 | 0.39 | >50 | 25 |
| Polyionene | 1849 | 0.78 | 6.25 | 3.125 | >50 | 0.78 |

The polyionene was about three times less toxic than the antibiotic and had acceptable antimicrobial activity.

Those skilled in the art will recognize or be able to ascertain using no more than routine experimentation many equivalents to the specific embodiments of the invention described herein. Such equivalents are intended to be encompassed in the scope of the following claims.

What is claimed is:

1. A method of preventing or inhibiting colonization or preventing or treating infection in a cystic fibrosis patient comprising the step of administering to said patient an effective amount of an ionene polymer.

2. A method of preventing or inhibiting colonization or preventing or treating infection in a cystic fibrosis patient comprising the step of administering to said patient an effective amount of an ionene polymer characterized by a repeat unit having the formula: 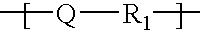
wherein R₁ is a substituted or unsubstituted hydrocarbyl group; and each Q is independently: 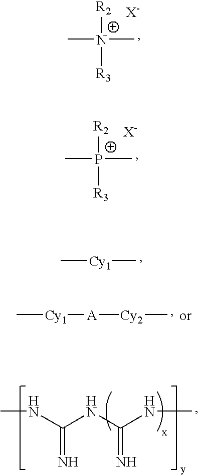
wherein:
Cy₁ and Cy₂ are each independently a quaternary nitrogen-containing monocyclic heteroaromatic ring or non-aromatic heterocyclic ring;
A is a covalent bond, or a substituted or unsubstituted lower alkylene group;
R₂ and R₃ are independently —H, a substituted or unsubstituted aliphatic or aromatic group;
each X⁻, separately or taken together with other X⁻s, is a physiologically acceptable anion;
x is an integer from 0-4; and
y is an integer from 1-5.

3. The method of claim 2, wherein said ionene polymer is administered therapeutically.

4. The method of claim 2, wherein said ionene polymer is administered prophylactically.

5. The method of claim 2, wherein R₁ is a substituted or unsubstituted arylene or lower alkylene group.

6. The method of claim 2, wherein the patient is at risk of pulmonary colonization or is suffering from a pulmonary infection.

7. The method of claim 6, wherein the polymer is administered by pulmonary means.

8. The method of claim 7, wherein the colonization or infection is caused by a microbe selected from the group consisting of Pseudomonas, Staphylococcus, Haemophilus, Streptococcus, Burkholderia, Aspergillus, Candida, Mycobacteria, Mycoplasma, Stenotrophomonas, Escherichia, and Klebsiella species, and combinations thereof.

9. The method of claim 8, wherein the microbe is selected from the group consisting of Pseudomonas aeruginosa, Staphylococcus aureus, Haemophilus influenzae, Burkholderia cepacia, Aspergillusfumigatus, Candida albicans, Mycoplasma pneumoniae, Stenotrophomonas maltophilia, Escherichia coli, Klebsiella pneumoniae, and combinations thereof.

10. The method of claim 7, wherein each R₂ and R₃ are each independently an alkyl group or a hydroxyalkyl group.

11. The method of claim 7, wherein said repeat unit has the formula:

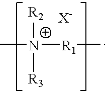

12. The method of claim 11, wherein R₁ is a substituted or unsubstituted straight chained lower alkylene group or polyalkylene glycol optionally substituted with one or more —OH groups.

13. The method of claim 7, wherein said repeat unit has the formula: 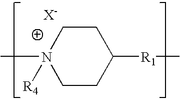
wherein R₄ is hydrogen or a substituted or unsubstituted lower alkyl group.

14. The method of claim 13, wherein R₄ is a lower alkyl or hydroxy substituted lower alkyl.

15. The method of claim 7, wherein said repeat unit has the formula: 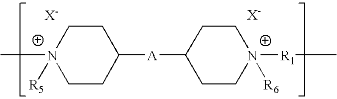
wherein A is a bond or substituted or unsubstituted lower alkylene group, and wherein R₅ and R₆ are each independently hydrogen or a substituted or unsubstituted lower alkyl group.

16. The method of claim 15, wherein R₅ and R₆ are each independently an alkyl group or a hydroxyalkyl group.

17. The method of claim 16, wherein A is an unsubstituted straight chained lower alkylene group.

18. The method of claim 17, wherein R₁ is a substituted or unsubstituted straight chained lower alkylene group or polyalkylene glycol optionally substituted with one or more —OH groups.

19. The method of claim 18, wherein R₁ is an unsubstituted polyalkylene glycol or —CH₂CHOH(CH₂)ₙCHOHCH₂— wherein n is an integer from 0 to 8.

20. The method of claim 7, wherein said repeat unit has the formula: 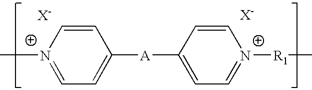
wherein A is a bond or substituted or unsubstituted lower alkylene group.

21. The method of claim 20, wherein A is an unsubstituted straight chained lower alkylene group.

22. The method of claim 21, wherein the repeat unit is represented by the formula:

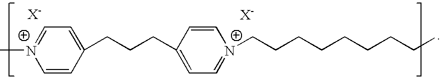

23. The method of claim 21, wherein R₁ is a substituted or unsubstituted straight chained lower alkylene group or polyalkylene glycol optionally substituted with one or more —OH groups.

24. The method of claim 23, wherein R₁ is an unsubstituted polyalkylene glycol or —CH₂CHOH(CH₂)ₙCHOHCH₂— wherein n is an integer from 0 to 8.

25. The method of claim 24, wherein the repeat unit has the formula:

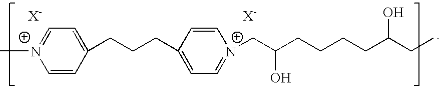

26. The method of claim 7, wherein said polymer is characterized by repeat units of the formula:

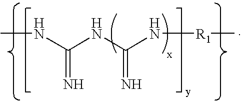

27. The method of claim 26, wherein said copolymer is characterized by the formula:

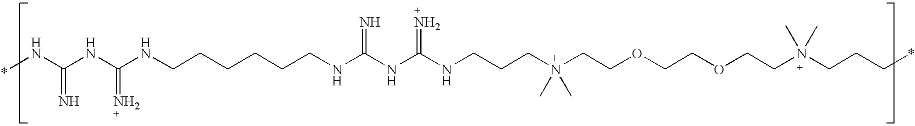

28. The method of claim 26, wherein one end or both ends of the polymer or copolymer are capped with a group represented by the formula: 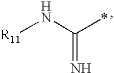
wherein R₁₁ is a C₂-C₉₀ alkyl, C2-C90 oxyalkyl, or aromatic group and the symbol “*” represents the bond connecting the cap to the polymer or copolymer.

29. A method of preventing or inhibiting colonization or preventing or treating infection in a cystic fibrosis patient comprising the step of administering to said patient an effective amount of an ionene copolymer characterized by a repeat unit having the formula: 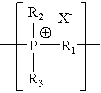
and a repeat unit of the formula: 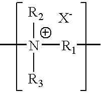
wherein:
R₁ is a substituted or unsubstituted hydrocarbyl group;
R₂ and R₃ are independently a substituted or unsubstituted aliphatic or aromatic group; and
each X⁻ in the polymer or copolymer, separately or taken together with
other X⁻s, is a physiologically acceptable anion.

30. The method of claim 29, wherein the patient is at risk of pulmonary colonization suffering from a pulmonary infection.

31. The method of claim 30, wherein the polymer is administered as an aerosol.

32. The method of claim 31, wherein the colonization or infection is caused by a microbe selected from the group consisting of Pseudomonas, Staphylococcus, Haemophilus, Burkholderia, Aspergillus, Candida, Mycobacteria, Mycoplasma, Stenotrophomonas, Escherichia, or Klebsiella species, and combinations thereof.

33. The method of claim 29, wherein said polymer or copolymer is comprised of repeat units of the formula: 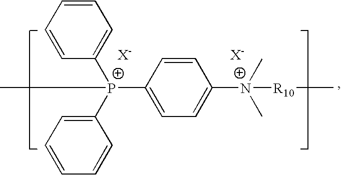
wherein R₁₀ is a substituted or unsubstituted lower alkylene group having from about 4 to about 12 carbon atoms and each X⁻, separately or taken together with other X⁻s is a physiologically acceptable anion.
